(19) United States
(12) Patent Application Publication
(10) Pub. No.: US 2005/0100563 A1
(43) Pub. Date: May 12, 2005

(54) Antifungal compositions and methods of treatment therewith

(76) Inventor: Don L. Hexamer, Dallas, TX (US)

Correspondence Address: Irving M. Fishman, 89 Headquarters Plaza, Suite 1422, North Tower, Morristown, NJ, 07960, US

(21) Appl. No.: 10/629,234

(22) Filed: Jul. 28, 2003

Related U.S. Application Data
(63) Continuation-in-part of application No. 09/770,336, filed on Jan. 26, 2001, now Pat. No. 6,676,953.

(51) Int. Cl. A61K 9/00

(52) U.S. Cl. 424/400

(57) ABSTRACT

The invention is an aqueous or aqueous/alcoholic solution of a transition metal halide, a transition metal oxyhalide, and/or the hydrolysis products thereof as an antifungal composition and method of treating fungal infections therewith. The composition may be used for humans and/or animals in need of topical antifungal treatment. The invention is particularly adapted to use for onychomycotic infections. The transition metal is selected from the groups consisting of copper, zinc, gallium, germanium, silver, tin and mixtures thereof (mixed metal compounds such as [SnZnF₄]), which can be in any oxidation state (other than that of the totally reduced metal). The halide is selected from the group consisting of fluorine, chlorine, bromine, iodide, and mixtures thereof (a mixed halide such as in [SnF₂Cl]⁻). The transition metal compounds may also be mixed oxides and halides such as in stannous oxyfluoride (Sn₄OF₆) or stannic oxyfluoride (SnOF₂). Since the compounds are in solution, they may also exist as hydrolysis products of any of the foregoing. The compositions have a pH of about 2 to about 4.

### CROSS-REFERENCE TO RELATED APPLICATIONS

This application is a continuation-in-part of U.S. application Ser. No. 09/770,336, filed Jan. 26, 2001.

### STATEMENT REGARDING

### FEDERALLY SPONSORED RESEARCH OR DEVELOPMENT

Not Applicable

### FIELD OF THE INVENTION

This invention relates to topical formulations having anti-fungal and/or anti-yeast infection properties. The invention is particularly suitable for use in the treatment of fungal or yeast infections of the fingernails, toenails, and the tissues beneath or surrounding the fingernails or toenails. The invention is also particularly suited for use in the treatment of fungal and/or yeast infections of mucosal membranous areas of the body, especially those of the mouth, nose and vaginal tract. The invention also relates to the treatment of fungal or yeast infections of the skin on other parts of the body. The invention also relates to transition metal (especially copper, gallium, germanium, indium, iron, nickel, silver, tin, titanium, zinc, and zirconium) halides, oxyhalides, and the hydrolysis products of each. The invention relates to both human and veterinary uses.

### BACKGROUND OF THE INVENTION

Fungal and yeast infections of the skin and nails are common in humans. Typical topical fungal infections include those generally in the stratum corneum, squamous mucosa, or cornea. These typically include dermatophytosis (ringworm), candidiasis, tinea versicolor, piedra, tinea nigra, fungal keratitis, onychomycosis (mycoses of the nails), and tinea capitis (mycosis of the hair). According to Goodman and Gilman's The Pharmacological Basis of Therapeutics, 9^{th} Ed. (McGraw Hill, New York, N.Y. 1996), p. 1185-1188 and 1606-1607, topical treatment with antifungal agents is usually not successful for mycoses of the nails and hair and has no place in the treatment of subcutaneous fungal infections.

A wide range of materials have been used in the past for the topical treatment of fungal and yeast infections and include gentian-violet, carbol-fuchsin, acrisocin, tracetin, sulfur, iodine, and aminacrine, most of which have fallen out of disuse in the fight against these dematological conditions. Current medical formulations include as the active agent imidazoles and triazoles (such as clotrimazole, econazole, ketoconazole, miconazole, terconazole, butoconazole, tioconazole, oxiconazole, and sulconazole), ciclopirox olamine, haloprogin, naphthyl compounds (such as tolnaftate, naftifine, and, terbinafine), polene antifungal antibiotics (such as nystatin and amphotericin B), undecylenic acid, benzoic acid, propionic acid, and caprylic acid. (See Goodman and Gillman p. 1185-1188; and Harrison's Principles of Internal Medicine 13^{th} Ed., (McGraw-Hill, New York 1994) p. 854-864.) However, none of these agents is indicated for topical use in the treatment of fungal infections of the nails (onchyomycoses) or for subcutaneous fungal infections of the skin. Used topically, these compounds are indicated for the more superficial skin infections. Onchymycoses and subcutaneous fungal infections have required systemic therapy with an antifungal agent.

Fungal infections in, under, and around the fingernails and toenails can be painful, unattractive, and are difficult to treat. Such fungal infections, variously referred to as onychomycosis, ringworm of the nails, and tinea unguinium, can cause thickening, roughness and splitting of the nails. Organisms known to play parts in fungal infections include species within the genuses Epidermophyton, Trichophyton, Malassezia, Micosporum, Pityrosporum, and Candida. Especially noted for their involvement in onychomycosis are Epidermophyton floccosum, Trichophyton rubrum, Trichophyton mentagrophytes, Trichophyton mengninii, Trichophyton schoenleinii, Trichophyton tonsurans, Microsporum canis, Microsporum audouinii, Microsporum gypseum, Pityrosporum orbiculare, Candida albicans, etc.

In addition to these infections of the nails and the surrounding tissues, infections due to these organisms and others can also present as superficial skin and/or mucous membrane infections. Exemplary fungal infections of this type include, without limitation, Candida, Tinea cruris and Tinea pedis, among others.

Furthermore, other fungal infections (including systemic fungal infections) often present with skin and/or mucous membrane symptoms such that topical treatment may be desired to accompany systemic treatment for the main infection locus. Such infections include: blastomycosis, paracoccidioidomycosis, pseudallescheriasis, sporotrichosis, crytococcosis, aspergillosis, etc.

As noted previously, while there have been a significant number of topical medications for the treatment of fungal infections, none has really been indicated for the treatment of the fungal infections of the nails and the tissues surrounding and below the nail. The treatments that have been available for such infections have traditionally been systemic, in particular oral or intravenous, which introduce concerns about systemic side effects and toxicity, and in the case of intravenous administration, inconvenience for long term dosage administration.

U.S. Pat. No. 5,916,545 discloses an antifungal composition of tioconazole, water, alcohol, and a gel former. A preferred composition comprises 15-30% tioconazole, 1-10% water, 50-90% alcohol, 0.5 of 5% of a gel forming agent, and a plasticizer.

U.S. Pat. No. 5,696,105 discloses a topical composition for the treatment of onychomycosis comprising an effective amount of mometasoneo furoate in a hydroalcoholic base comprising 15-50 weight % propylene glycol, 20-40% isopropyl alcohol, 20-60% water, 0.1-3 % thickening agent, and buffer, sufficient to adjust the pH to between 3.0 and 6.0, preferably between 4.0 and 5.0.

U.S. Pat. No. 5,972,317 discloses a method for treating diseased nails with a nail-permeable topical composition comprising a medicament and a proteolytic enzyme.

U.S. Pat. No. 5,486,537 discloses an antifungal composition comprising a solution of cinnamic aldehyde, up to about 20 weight % griseofulvin and at least 5 volume % of an alcohol selected from ethanol and isopropanol for topical application to skin and keratinous tissue.

U.S. Pat. No. 4,919,920 discloses a topical composition for hardening nails, the composition comprising a cosmetically acceptable aqueous vehicle including an effective amount (for nail hardening) of fluoride ion and having a pH of about 3.5 to 8. The fluoride ion is from a fluoride compound selected from water soluble fluoride salts and complex fluoride salts, including aluminum fluoride, sodium fluoride, potassium fluoride, stannous fluoride, stannous monofluorophosphate, etc.

U.S. Pat. No. 5,395,241, U.S. Pat. No. 5,057,309, and U.S. Pat. No. 3,346,578 disclose the use of stannous fluoride in oral hygiene preparations. U.S. Pat. No. 4,267,164 provides an effervescent tablet of stannous fluoride and shows in the examples a particular one that upon dissolution results in a 0.4% stannous fluoride solution.

U.S. Pat. No. 4,599,228 discloses stannous fluoride compositions in combination with other active agents. The specifically disclosed compositions are anhydrous preparations. U.S. Pat. No. 5,663,208 discloses antifungal compositions having antifungal agents and in addition stannous fluoride as a conventional additive. Up to 1% solutions of the fluoride are mentioned, with 0.1% being preferred.

U.S. Pat. No. 5,747,070 discloses stannous halides for use as anti herpes virus composition. When fluorine is the halide, another halide must also be present. U.S. Pat. No. 6,098,716 teaches anhydrous formulations of stannous fluoride as an antiviral.

U.S. Pat. No. 6,200,553 teaches stannous fluoride solutions in amounts of 0.5% to 5% as nail hardening compositions for use in combination application with a calcium solution. In practice, the calcium solution is applied and then the fluoride solution is applied. The resultant effect is that the calcium binds to the nail and then the fluoride binds to the calcium. The calcium-fluoride complex is what makes the nail harder. Effectively, the calcium pulls the fluorine out of the solution so that there is little or no fluoride ion left in solution.

U.S. Pat. No. 6,248,370 discloses an aqueous shampoo having selenium and zinc active in which stannous fluoride is also present.

U.S. Pat. No. 5,908,640 and U.S. Pat. No. 6,455,076 disclose stannous fluoride solutions for the purpose of preventing skin irritation.

U.S. Pat. No. 4,828,822 discloses background information on the instability of stannous fluoride.

All of the foregoing U.S. patents are incorporated by reference herein in their entirety.

### OBJECTS OF THE INVENTION

It is among the objects of the present invention to provide an antifungal topical preparation that is capable of treating an onychomycotic infection.

It is another object of the invention to provide an inorganic material that effectively treats fungal infections in the skin, mucous membranes, and in and under nails.

It is still a further object of the invention to provide an antifungal transition metal halide product.

It is still a further object of the invention to provide an antifungal transition metal fluoride product.

It is still a further object of the invention to provide an antifungal product having at least one compound having (a) a metal component of Ag, Cu, Fe, Ga, Ge, In, Ni, Sn, Ti, Zn, and/or Zr and (b) a halide component of fluoride, chloride, bromide, and/or iodide.

It is yet a further object of the invention to provide a kit comprising an antifungal material, along with instructions for preparing a proper antifungal solution therewith, and instructions for use thereof in the treatment of fungal infections.

Still another object of the invention is to provide a method for the topical treatment of fungal infections.

Still other objects of the invention will be recognized by those of ordinary skill in the art.

### BRIEF SUMMARY OF THE INVENTION

These and other objects of the invention are surprisingly achieved by applying an aqueous or aqueous/alcoholic solution of a transition metal halide, a transition metal oxyhalide, and/or the hydrolysis products thereof to an area of the human or animal body in need of antifungal treatment. The transition metal is selected from the group consisting of Ag, Cu, Fe, Ga, Ge, In, Ni, Sn, Ti, Zn, Zr and mixtures thereof (mixed metal compounds such as [SnZnF₄]), which can be in any oxidation state (other than that of the totally reduced metal). The halide is selected from the group consisting of fluorine, chlorine, bromine, iodide, and mixtures thereof (a mixed halide such as in [SnF₂Cl]⁻). The transition metal compounds may also be mixed oxides and halides such as in stannous oxyfluoride (Sn₄OF₆) or stannic oxyfluoride (SnOF₂). Since the compounds are in solution, they may also exist as hydrolysis products of any of the foregoing. Alternatively, the transition metal compound can be replaced, in part or totally by HF, with the pH optionally adjusted into the range set forth below by virtue of an appropriate amount of a pH adjusting agent. The compositions have a pH of about 2 to about 4.

The present invention composition is an antifungal composition preferably comprising water; optionally a wetting agent such as a lower alkyl, monohydric or dihydric alcohol; a transition metal halide compound that on water dissolution is able to provide fluoride ion to the solution, where the pH upon dissolution is naturally in the range of about 2.0 to about 4.0.

The compositions of the invention are particularly suited for the treatment of fungal infections of the toenails and fingernails (or similar keratinaceous body parts in animals). They are desirably aqueous, fast drying, and non-greasy. Such compositions are preferably easy to apply topically, will readily penetrate around and under a nail and into surrounding and underlying skin, and will desirably control or eradicate the offending fungal organism without damaging the skin or healthy tissue. For the same reasons, the present invention is also suited for the treatment of subcutaneous fungal infections. The invention may also be used for treating fungal infections of other portions of the skin and/or mucous membranes.

In addition to the treatment of fungal infections, the invention is also suited to the treatment of other conditions such as acne, aphthous ulcers, bullous pemphigoid, carbuncles, chiggers, folliculitis, furuncle, herpes simplex, herpes zoster (including shingles), impetigo, lyme disease, molluscum contagiosum, pfiesteria, pimples, pityriasis rosea, psoriasis, scabies, and seborrheic dermatitis or seborrhea. For these and other non-onychomycotic infections, various formulations having the active agent set forth above (in essentially the same concentrations as set for above for fungal infections) can be used. Such formulations include simple solutions of the active agent in water or non-aqueous liquid media, suspensions, lotions, creams, gels, etc and the formulations may be delivered in a variety of methods, such as direct application by the hand, fingers or delivery media (such as cotton swab or impregnated swabs, cloths, or bandages; or via topical or transdermal patches containing a suitable formulation therein.

### BRIEF DESCRIPTION OF THE DRAWING

Not Applicable

### DETAILED DESCRIPTION OF THE INVENTION

The preferred compositions of the invention comprise water, optionally a lower monohydric or dihydric alcohol, an antifungal transition metal halide or HF or combination thereof in a suitable concentration such that on dissolution in the water or water/alcohol system, the pH is in the range of about 2.0 to about 4.0, and the solution is antifungally active. While not being bound to theory, the following theories are proposed, and any one or all may be operating at the same time:
(1) the solution provides a dilute solution of HF where the fluoride ion and/or the H ion or both together provide the ability of the active agent to penetrate to the source of the infection and the pH which results is one that the fungal organisms cannot tolerate;
(2) the transition metal ion and/or one of its complexes (halide, oxyhalide, or hydrolysis products of either) in solution is poisonous to the fungus per se or at the pH induced by the solution at the locus of the infection.

It is believed that both mechanisms are at play and that the transition metal poisoning of the fungus supplements the action of the halide ion and the pH. The theories are also intertwined in that when the transition metal compound is present, as it dissolves, hydrolysis reactions take place so that hydrogen ions as well as halide ion (inclusive of [halide]⁻, [M^{A+}(halide⁻)_{(A+Y)}]^{Y-}, and [(M^{A+})_{W}((halide⁻)_{(WA+Y)})]^{Y-}, [(M^{A+})_{W}((halide⁻)_{(WA-Y)})]^{Y+}) as well as hydrolysis product complex ions (one or more of the above further complexed with hydroxyl groups) results. In the above cases, the transition metal may be in any of its allowed oxidation states and any of these species may be present as the corresponding oxide oxidation species (two halogens in any of the above, but not all of them, being replaced by an oxygen). When the oxides are present, on dissolution, they may remain as the oxide or convert on hydrolysis to two hydroxyl groups.

The transition metal compounds for us in the present invention are typically selected from Ag, Cu, Fe, Ga, Ge, In, Ni, Sn, Ti, Zn, Zr, and mixtures thereof, the mixture being mixed metal compounds, such as ZnSnF₄ for example. Preferably, the transition metal is selected from zinc, tin, and mixtures thereof, with the most preferred being tin. Another preferred compound for use in the present invention is HF, although tin is still more highly preferred.

The transition metal compounds for use in the present invention are preferably halides selected from the group consisting of fluoride, chloride, bromide, iodide, and mixtures thereof, mixtures being mixed halides such as SnFCl, SnF₂ClBr, etc. Preferably, the halides are not mixed halides and the preferred halogen is fluorine. When mixed halides are used, it is preferably that at least one of the halogen atoms is fluoride. The term halide in the context of the present invention further includes the halophosphates such as halomonophosphate and hexahalophosphate (as in stannous monofluorophosphate, for example) among others. While these species are less preferable than the simple halogens without the phosphate group, they may nonetheless be utilized when desired.

The transition metal halides for use in the present invention can be present as oxyhalides such as SnOF₂ (the tin atom being in the +4 oxidation state) or Sn₄OF₆ (the tin being in the +2 oxidation state). Such compounds can be formed by allowing the SnF₂ to be exposed to oxygen and convert thereto, the former compound being an oxidation of the stannous fluoride and the latter compound being a replacement of two fluorine atoms by an oxygen atom without changing the tin oxidation state. Furthermore, excess halide results in higher solubility in the creation of species such as [SnF₃]⁻ and [Sn₂F₅]⁻ for example. Compounds of this nature can be prepared by dissolving the parent compound in insufficient water (thereby leaving some solids at the bottom of the dissolution vessel) and adding fluoride in the form of NaF or other suitable salt (typically an alkali metal halide), and isolating the Na⁺[SnF₃]⁻ for example. Alternatively, any of the foregoing species can be formed in situ. Similar transformations and species involving the other transitions metals and halides will be apparent to those of ordinary skill in the art. Furthermore, any of these, when added to water will form hydrolysis products, some of which can be prepared and isolated, other will only arise in solution. In either event, preparation of the hydrolysis product species and subsequent dissolution as well as dissolution of the compounds that are not the hydrolysis products, but give rise to the hydrolysis products as set forth above are both contemplated as being with in the invention. As such, the general formula for the transition metal halide compound may be:
[(Q⁺)_{Y}(M^{A+})_{W}((halide⁻)_{(WA+Y-2Z-P)})(O)_{Z}(OH)_{P}],
where Q is an alkali metal, preferably sodium or potassium, or Q is hydrogen, M is the transition metal set forth above, A is the valence of the transition metal, Z, and P are each independently selected from 0 to the maximum coordination number available for the molecule provided that Z+P does not exceed the maximum coordination number, and W is an integer of from 1 to the maximum number of transition metal atoms that can associate together in a cluster or polymeric particle (that can on dissolution yield an effective antifungal composition) and Y is a number from 0 to the maximum number of halide ions that can associate to form a soluble species with the transition metal M, provided that Y does not exceed the coordination number of the M cluster or polymer. The coordination number for stannous chloride and stannous bromide are 9 and 8 respectively (See Cotton et al Ed.; Advanced Inorganic Chemistry, (John Wiley & Sons, NY 1999), p. 301-303). Preferably the transition metal is zinc or tin, most preferably tin. Preferably the halogen is fluorine, chlorine, bromine, or iodine; more preferably fluorine or chlorine, most preferably fluorine. Preferably, there is only one transition metal present per molecule (other than minor contaminants that might not otherwise be removable). Preferably there is only one halogen atom present per molecule. Where there is more than one transition metal present, preferably tin is one of the transition metals. Where there is more than one type of halogen present, preferably at least fluorine is present. Exemplary transition metal halide compound species in solution when using tin as the transition metal and fluorine as the halide can include the following stannous species [SnF₃]⁻; [Sn₂F₅]⁻; [SnF]⁺; [Sn(OH)]⁺: [Sn₂(OH)₂]⁺²; [Sn₃(OH)₄]⁺²; Sn₃O₂(OH)₂; and Sn₄OF₆ among others (see A. Vogler et al., Inorg. Chem. 1992, 31, 3277; Donaldson et al, J. Chem. Soc. Dalton Trans. 1995, 2273; and Abrahams et al., J. Chem. Soc. Dalton Trans. 1994, 2581) and stannic species such as [Sn(OH)₆]⁴⁻, and [SnF₆]²⁻ and other intermediary complexes having both OH and F ligands. Still other species such as [Sn(OH)₂]⁰; [Sn(OH)₃]⁻; [Sn₂(OH)₃]⁺; [Sn(OH)₅]⁻; [SnO₃]²⁻; [SnF]⁺; [SnF₃]⁻; [SnF₆]²⁻; etc are discussed in Seby et al, Geochimica et Cosmochimica Acta, Vol. 65, No. 18, 2001, pp 3041-3053.

Most preferably the transition metal halide is stannous fluoride, an oxidation product of stannous fluoride, and/or hydrolysis products of one or both. Stannous fluoride is known to be extremely sensitive to atmospheric oxygen and to moisture, and unless protected, converts (at least in part) to stannic oxyfluoride and other oxidation species even in the substantially dry state. In a most highly preferred embodiment, stannous fluoride is allowed to be exposed to atmospheric conditions to allow for the partial to complete conversion thereof to an oxidized form, preferably stannic oxyfluoride, which is then used to prepare the preferred formulation.

Some compounds mentioned in the literature and considered within the scope of the invention include, without limitation: potassium stannous fluoride (U.S. Pat. No. 2,606,812); stannous chlorofluoride (U.S. Pat. No. 2,836,544); distannous monochlorotrifluoride (U.S. Pat. No. 2,882,204); stannous fluorozirconate (U.S. Pat. No. 3,266,996). Each of these patents is incorporated by reference herein in its entirety.

The formulations of the invention are an effective antifungal amount (preferably an effective onychomychotic treating effective amount) of (a) the transition metal halide compound, (b) HF, or (c) a combination thereof, in a concentration sufficient to provide a pH of about 2 to about 4, preferably about 2.25 to about 3.75, more preferably about 2.5 to about 3.5, still more preferably about 2.75 to about 3.25, most preferably about 2.8 to about 3.2; and a halide content of about at least about 3 mmole halide/100 ml of solution (0.06 grams fluorine/100 ml solution when fluorine is the halide), preferably at least about 3.25 mmole halide/100 ml, more preferably at least about 3.5 mmole halide/100 ml, most preferably at least about 3.8 mmole halide/100 ml; and up to the maximum solubility of the compound, but preferably in a concentration that if prepared as a simple solution in water would be a concentration of transition metal halide that is less than isotonic, preferably not more than about 80% of isotonic, more preferably not more than about 50% of isotonic, even more preferably not more than about 22% of isotonic, yet more preferably not more than about 21% of isotonic, most preferably not more than about 20% of isotonic. Where transition metal non-halide compounds are used in conjunction with non-transition metal halide sources, the non-transition metal halide is used in an amount to provide at least the minimum halide content and the transition metal non-halide compound is used in amounts of at least about 1.5 mmole/100 ml, preferably at least about 1.6 mmole/100 ml, more preferably at least about 1.75 mmole/100 ml, most preferably at least about 1.9 mmole/100 ml, and the combined transition metal maximum concentrations of the transition metal non-halide compound and the non-transition metal halide source is such that in a simple water solution thereof, the halide ion and transition metal ion content are together less than isotonic, preferably not more than about 80% of isotonic, more preferably not more than about 50% of isotonic, even more preferably not more than about 22% of isotonic, still more preferably not more than about 21% of isotonic, most preferably not more than about 20% of isotonic. For compounds having maximum solubility near the lower limits set forth above or within the ranges set forth above, additional solids can be and preferably are added to the solutions. This is especially so for compounds having solubilities near the lower limits set forth above. For example, in such situations, it is preferable to prepare a saturated solution in water (evidenced by undissolved materials after a reasonable time for equilibration), filtering or decanting the liquid portion, and adding a small portion of additional transition metal halide compound to the liquid. If the pH of the resulting solution is outside of the required range, then a pH adjuster can be added (acid, base, or buffer) to bring the pH to the desired level. For compounds that are too insoluble to reach the fluorine content, additional fluoride can be added in the form of alkali metal fluoride, which results in solubilizing the compound further through formation of transition metal halide compound-halide ions, such as in ([Na]⁺)₂ [SnF_{6]}²⁻ from the addition of sodium fluoride to stannic fluoride. Alternatively HF may be added if the compound used provides insufficient fluoride and the pH is too high. In another variation, the transition metal component and the halide component can be completely contributed by different starting materials however, this is generally less desirable as it introduces additional counterions from each compound making the composition more complex and increasing the risk of incompatibilities. Nonetheless, when desired, the formation of the ionic mixture in solution can be made in this way in situ when desired. This variation is especially useful if the particular transition metal halide desired is difficult to prepare as a pure compound. Various modifications on the theme will be apparent to those of ordinary skill in the art.

Where a pH modifier is desired, any inorganic or organic acid or base or buffer may be used that is not incompatible with the remainder of the composition components and is pharmacologically acceptable consistent with the particular end use. Hence, where the end use is to be for a mucosal application, the pH modifiers should be pharmaceutically acceptable for application to mucosal membranes. Where the end use is for strictly external applications, the pH modifier may be selected from a broader class provided the acid, base, or buffer used does not substantially penetrate the skin and reach the blood stream. In the case of the pH modifier which does migrate into the tissues along with the active agents, the pH modifier should be selected from those that are systemically acceptable. Hence, systemically acceptable pH modifiers are preferred. Non-limiting exemplary inorganic pH adjusters and buffers include: hydrohalides such as HF, HCl, HBr and HI (especially when the halide is the same as that of the transition metal halide used); sulfuric acid; nitric acid; phosphoric acid; among others; as well as the salts of those acids having more than one ionizable hydrogen. Non-limiting exemplary organic acids include mono, di, and poly carboxylic acids and hydroxyacids, such as acetic, propionic, maleic, fumaric, malonic, malic, citric, lactic, tartaric, etc. (as well as their respective salts, especially alkali metal salts). Non-limiting organic acids having acidic groups that are not carboxylic acids may also be used such as sulfonic acids and phosphonic acids (as well as their respective salts). pH adjusters which also act as chelators for the particular transition metal are less preferred in that it promotes larger species making the migratin of the transition metal species more difficult. In some instances, the chelation may be so strong that the intrinsic effectiveness of the transition metal halide compound is compromised. As such, the pH modifiers are preferably either inorganic or mono or di organic acids, more preferably inorganic or mono organic acids. For certain species even the di organic acids may chelate two tightly and for such species, the pH adjuster should be inorganic or a mono organic acid. Specific preferences are well known in the art and will be chosen based on the specific chemistry of the transition metal compound being used. For essentially the same reasons, chelators (where pH adjusters or not) are preferably excluded from the compositions of the invention.

The above solution may be applied to the affected area to treat the fungal infection topically. Since topical treatment of subcutaneous infections as well as those in or under the nails requires substantial penetration of the solution into the affected tissues, it is preferable to further include a wetting agent/penetration enhancer, typically a lower monohydric or dihydric alcohol having 2-3 carbon atoms, more preferably, ethanol, isopropanol, or propylene glycol. Other penetration enhancers known in the transdermal arts may also be used, but they are less preferred embodiments. If desired, surfactants may also be utilized to aid in the wetting/penetration of the tissues by the invention solution. When the wetting agent/penetration enhancer is an alcohol, the composition may contain up to about 80% by volume of alcohol, preferably up to about 60%, more preferably up to about 60%, still more preferably up to about 40%, even more preferably up to about 20%, even more preferably up to about 15% of said alcohol. While there is no minimum requirement for the wetting agent, when the product is designed to be used for onychomycotic infections or subcutaneous infections, the wetting agent is preferably present, and when present is preferably present in an amount of at least about 3 volume %, preferably at least about 5 volume %, still more preferably about 10 volume %, even more preferably at least about 14 volume %. In calculating the alcohol volume %, the neat alcohol is the amount to be considered and the entire formulation is the basis. Hence, if 70% isopropanol is to be used and a 14 volume % is desired, then 20 mls of 70% isopropanol is added to 80 mls of the previously prepared solution (transition metal halide in water) to achieve the 14 volume % alcohol.

Generally, the use of thickeners is contrary to the purpose of having a wetting agent present. As such, thickeners are generally excluded from the composition intended for use in skin and nail fungal treatment compositions. In cases where the treatment is of mucosal membranes, the composition is not intended to be in prolonged contact with the mucous membrane so as to avoid excessive absorption of the transition metal compound. As such, thickened solutions are generally not desirable and thickeners are to be generally excluded from those compositions. In each of the above cases, a material which has thickening properties but does not substantially thicken the composition in question can be included for other formulation purposes, but only in amounts that do not substantially increase the composition viscosity. The one exception to this is in the use of a composition of the invention for the treatment of the oral mucosa and other oral tissues. In such a situation, a slightly thickened composition can be employed since rinsing the mouth after the appropriate exposure can be readily accomplished. Nonetheless, even in compositions intended for treating the oral mucosa and other oral tissues, it is preferably to exclude thickeners in any amounts that substantially increase the viscosity over that of the composition without the thickener. In the most preferred embodiments, thickeners are totally excluded from the invention compositions.

Other materials that are preferably excluded from the invention compositions include skin soothing agents, resins, phytic acid and its salts, siloxane polymers, sanguinarine, and selenium compounds. It is also preferable that the transition metal halide compound and/or HF be the only antifungally active agent in the composition. Compounds used for irritant purposes are to be excluded from the compositions.

Once the solutions are prepared, the composition should be placed in containers in small amounts with (in the case of those composition near the lower end of the fluoride content, a small amount of additional solid material. In the case of compositions near the lower limit of pH, users should be cautioned to keep the preparation tightly closed, to minimize evaporation which could result in the pH falling below the lower limit. While the composition will still be effective at these lower pHs, excessively low pH can be hazardous. Therefore, it is preferable to have a composition that is at the more preferred pH ranges set forth above.

Other components that may be desirable and do not affect the active agent transition metal halide compound, draw the pH out of the desired range, or affect the ability of the composition to reach the locus of the fungal infection may be added as long as they are consistent with the intended use (mucosal membrane treatment must not contain materials that are inconsistent with application to mucous membranes). Such materials include formulation aids, stabilizers, solubility enhancers, “skin feel” modifying agents, colors, fragrances, etc., which are well known in the pharmaceutical and personal care products arts.

The compositions of the invention may also be in dry form for reconstitution with a given amount of carrier, which may be provided in a separate container. In such a case, solid may be provided as a powder or other solid dosage form (such as an effervescent tablet or rapidly dissolving tablet for example) having the transition metal halide alone or with some or all of the other solid components of the formulation and a given amount of the carrier (water and any of other components not present along with the transition metal halide compound) separately provided. In such a case, either the patient, the pharmacist, or a patient care provider mixes the liquid carrier with the powder to reconstitute the composition shortly before use. The advantage of this type of dispensing of the composition is that the shelf life of the composition doesn't begin to run until reconstitution.

Once the composition is prepared, and ready for administration, the patient or caregiver, applies a small amount of the composition to the affected area. In the case of infections of the nails and the underlying tissues, the application is to the nail and the surrounding tissues and as can best be applied in and around the nailbed. In severe cases, the application is initially once a day until the infection begins to abate, after which less frequent administration is acceptable. Preferably administration is once weekly until the infected area has effectively treated. Treatment is preferably continued until the infected portion of the nail or skin has grown out and been sloughed or trimmed off. In the treatment of non-onychomychotic infections and conditions (whether or not of fungal origin) such as psoriasis, acne, and other bacterial, viral and fungal infections not associated with the nails, the formulation preferably does not contain an alcohol component, but may have such, if desired. In such cases where the alcohol free solution is desired, these non-onychomycotic conditions are suitably treated by utilizing the initial water solution of the active agent of formula I above (i.e. before any alcohol is added). For these non-onychomycotic conditions, compositions of the invention may include those materials that are desirably absent from the onychomycotic condition treating compositions mentioned above (namely: thickeners, skin soothing agents, resins, phytic acid and its salts, siloxane polymers, sanguinarine, and selenium compounds).

Where compositions are intended for use on the oral mucosa, a small amount of the composition may be rinsed throughout the mouth on a regular basis for several seconds up to about 1 minute on a frequency of once-twice daily initially, to once weekly for maintenance until the infection is resolved. The solution should not be swallowed.

For treatment of other mucosal membranes (nasal, vaginal, rectal) appropriate auxiliaries or adjunct formulations can be used to insure appropriate dosing. For example, vaginal application can be in the form of a douche type solution, followed shortly thereafter by a rinsing, cleansing douche. Rectal administration could be followed by a cleansing enema.

For treatment of the external dermatological conditions mentioned above (non-mucosal membranes) other than conditions of the nails, such external conditions including bacterial, viral, other fungal infections, and other topical conditions such as (without limitation) acne, carbuncles, chiggers, folliculitis, furuncle, herpes simplex, herpes zoster, impetigo, pimples, pitytiasis rosea, psoriasis, scabies, seborrheic dermatitis, tinea cruris, tinea pedis, etc.) 3 to 4 drops of a solution of the invention is used to moisten an approximately 7.5 cm×7.5 cm affected area, which is generally simply applied without rubbing. The area is typically not washed for a a period of about 3 to 4 minutes, after which the area can be washed if desired. The application generally repeated twice a week until improvement of the condition is seen. In most instances, after 6 months, treatment can be stopped or reduced, depending upon the condition, its initial severity, and the degree of response.

Other variations on the specifically disclosed embodiments of the invention above will be apparent to those of ordinary skill in the art.

### EXAMPLES

While not limiting the scope of the invention, the following examples are presented to illustrate certain embodiments thereof.

### Example 1

### Tin Halide Solution

Stannous fluoride is obtained commercially from a repacker and stored without restriction as to exposure to atmospheric oxygen and moisture. A heaping teaspoon of the compound is placed in a 16 ounce container, which is then filled with water. The container is closed and hand shaken for a couple of minutes. Some solids remain on the bottom. (The solubility should be approximately 0.3%, in agreement with stannic oxyfluoride, as opposed to stannous fluoride which has a solubility of 30%). The composition is allowed to stand for at least one hour. Carefully pour off the liquid and add approximately 25% (by volume thereof) of 70% isopropyl alcohol (to result in a final solution having 20% of 70% isopropanol which is a final neat isopropanol concentration of 14 volume %). The mixture is then dispensed into ½ ounce bottles and a small amount (50-80 mg) of the compound (from the stored material that has been exposed to ambient atmosphere) is added to each bottle.

### Example 2

### Treating Infected Nails

Infected nails are treated by applying one drop per nail to all nails on the affected hand or foot, when the nails are dry. The solution is applied under the nail and allowed to penetrate further to the infection locus. The nails and surrounding tissues are not washed or otherwise wetted for at least 30 seconds. Weekly applications continue until the infected portion of the nail grows out entirely.

### Example 3

### Preventative Use For Nail Infections

For use as a preventative against recurrence after the infection has been treated, and/or concurrent preventative treatment of the nails on appendages other than the infected one(s), the solution in Example 1 is applied one drop per nail per month, when the nails are dry. The solution is applied under the nail and allowed to penetrate further to the infection locus. The nails and surrounding tissues are not washed or otherwise wetted for at least 30 seconds. Monthly applications may continue as desired.

### Example 4

### Composition for Treatment of Mucosal Infections

Stannous fluoride is obtained commercially from a repacker and stored without restriction as to exposure to atmospheric oxygen and moisture. A heaping teaspoon of the compound is placed in a 16 ounce container, which is then filled with water. The container is closed and hand shaken for a couple of minutes. Some solids remain on the bottom. (The solubility should be approximately 0.3%, in agreement with stannic oxyfluoride, as opposed to stannous fluoride which has a solubility of 30%). The composition is allowed to stand for at least one hour. The liquid is carefully poured off and a small amount of the compound (from the stored material that has been exposed to ambient atmosphere) is added to the liquid. (approximately 80 mg/ounce).

### Example 4

### Treatment of Oral Infections

Approximately 1 ounce of the composition in Example 3 is swished around the mouth once a day for a few seconds, without swallowing, and the entire amount is spit out. After an additional 10-15 seconds, the mouth is rinsed with several ounces of water and again, the entire amount is expectorated. The procedure is repeated daily until the infection is no longer noticeable.

1. A topical antifungal composition for the treatment of a fungal infection of the skin or nails, or the symptoms associated therewith, in an animal, inclusive of human beings, in need thereof, said composition being substantially free of skin soothing agents, skin irritants, resins, phytic acid and salts thereof, chelating agents of a first and second compound as set forth below, siloxane polymers, sanguinarine, selenium sulfide, and substantially free of a thickening effective amount of an organic thickener, said composition comprising:
(a) water; and
(b) an antifungally active member selected from the group consisting of
(i) a first compound capable of generating in aqueous solution, at least one type of halogen ion X and H ion;
(ii) a second compound capable of generating in aqueous solution at least one halide ion X but not a significant amount of H ions in combination with a third compound capable of generating in aqueous solution H ions but not a significant amount of at least one halide ion X; and
(iii) mixtures thereof;
wherein each of said first and second compound comprises a transition metal selected from the group consisting of Ag, Cu, Fe, Ga, Ge, In, Ni, Sn, Ti, Zn, and Zr; and said at least one halide ion X is selected from the group consisting of F, Cl, Br, I, halophosphates of each, and mixtures thereof;
(c) an alcohol selected from the group consisting of lower monohydric alcohols and lower dihydric alcohols, present in amounts of from 0% up to about 80% by volume based on the final formulation;
(d) optionally a pH adjuster component selected from acids, bases and buffers in amounts sufficient to maintain the pH of said composition in the range of 2.0 to 4.0;
(e) said composition having a pH of about 2.0 to about 4.0,
wherein said composition is substantially free of said organic thickening agents in effective thickening amounts, substantially free of said skin soothing agents, substantially free of skin irritants, substantially free of resins, substantially free of phytic acid and salts thereof, substantially free of chelating agents for said first compound, if said first compound is present, substantially free of chelating agents for said second compound, if said second compound is present, substantially free of siloxane polymers, substantially free of sanguinarine, and substantially free of selenium sulfide
whereby when said composition is applied to said skin or nail, or to tissue surrounding or tissue underlying said nail, said fungal infection or symptoms associated therewith are effectively ameliorated.

2. The composition of claim 1 wherein said first compound is selected from halides of the formula
[(Q⁺)_{Y}(M^{A+})_{W}((X⁻)_{(WA+Y-2Z-P)})(O)_{Z}(OH)_{P}]
where Q is an alkali metal counter ion, M is said transition metal, X is said halide, A is an oxidation state of the metal atom M, and Y is an integer where at least one of an X ion and M_{(>0 to W)} X_{(1 to WA+Y)} ion is a soluble species, or at least one of the oxides thereof or at least one of the hydrolysis products thereof is a soluble species, each of (WA+Y), Z, and P cannot exceed the coordination number for (M^{A+})_{W}, and W is a number of at least 1.

3. The composition of claim 1 wherein said first compound is present in an amount sufficient to saturate said composition.

4. The composition of claim 1 wherein said first compound is present in excess of its saturation in said composition.

5. The composition of claim 1 wherein said metal M is selected from the group consisting of zinc and tin.

6. The composition of claim 5 wherein said metal M is tin.

7. The composition of claim 1 wherein said halide is fluorine.

8. The composition of claim 2 wherein at least a portion of said compound is present as an oxide.

9. The composition of claim 2 wherein at least a portion of said compound is present as at least one hydrolysis product.

10. The composition of claim 2 wherein at least a portion of said compound is present as a mixed oxide/hydrolysis product.

11. The composition of claim 1 wherein said metal M is tin and said halide is fluoride.

12. The composition of claim 1 wherein said alcohol is selected from the group consisting of ethanol, isopropanol, propylene glycol, and mixtures thereof.

13. The composition of claim 1 wherein said alcohol is isopropanol.

14. The composition of claim 1 wherein component b is substantially the only antifungal component present in said composition.

15. The composition of claim 1 wherein component b is the only antifungal component present in said composition.

16. An antifungal composition for the treatment of a fungal infection of the skin or nails, or the symptoms associated therewith, in an animal, inclusive of human beings, in need thereof, comprising
(a) water; and
(b) a weak hydrofluoric acid solution generator compound or hydrofluoric acid wherein the hydrofluoric acid concentration resulting in the solution is in sufficient to yield a pH in the range of not less than about 2.0, and not greater than about 4.0;
whereby when said composition is applied to said skin or nail, or to tissue surrounding or tissue underlying said nail, said fungal infection or symptoms associated therewith are effectively ameliorated.

17. An antifungal composition for the treatment of a fungal infection of the skin or nails, or the symptoms associated therewith, in an animal, inclusive of human beings, in need thereof, comprising
(a) water; and
(b) a metallic compound which is toxic to fungal organisms without being toxic to mammalian skin and nails, wherein said metallic compound is selected from the group consisting of
(i) a first compound capable of generating in aqueous solution, at least one type of halogen ion X and H ion;
(ii) a second compound capable of generating in aqueous solution at least one halide ion X but not a significant amount of H ions in combination with a third compound capable of generating in aqueous solution H ions but not a significant amount of at least one halide ion X; and
(iii) mixtures thereof;
wherein each of said first and second compound comprises a transition metal selected from the group consisting of Ag, Cu, Fe, Ga, Ge, In, Ni, Sn, Ti, Zn, and Zr; and said at least one halide ion X is selected from the group consisting of F, Cl, Br, I halophosphates of each, and mixtures thereof;
said compound being present in an amount sufficient to result in a pH of about 2.0 to about 4.0 or said composition further comprising a pH adjuster to bring said pH into the range of about 2.0 to about 4.0;
whereby when said composition is applied to said skin or nail, or to tissue surrounding or tissue underlying said nail, said fungal infection or symptoms associated therewith are effectively ameliorated.

18. An antifungal composition for the treatment of a fungal infection of the skin or nails, or the symptoms associated therewith, in an animal, inclusive of human beings, in need thereof, comprising
(a) water; and
(b) a non-nail hardening but fungally toxic amount of a fluoride ion donor compound; said compound being present in an amount sufficient to result in a pH of about 2.0 to about 4.0 or said composition further comprising a pH adjuster to bring said composition into the pH range of about 2.0 to about 4.0;
whereby when said composition is applied to said skin or nail, or to tissue surrounding or tissue underlying said nail, said fungal infection or symptoms associated therewith are effectively ameliorated.

19. A non-nail hardening but antifungal composition for the treatment of a fungal infection of the skin or nails, or the symptoms associated therewith, in an animal, inclusive of human beings, in need thereof, comprising
(a) water; and
(b) a non-nail hardening but fungally toxic amount of a fluoride ion donor compound;
whereby when said composition is applied to said skin or nail, or to tissue surrounding or tissue underlying said nail, said fungal infection or symptoms associated therewith are effectively ameliorated without substantially hardening of said nail.

20. A method of treating a fungal infection of at least one of the skin and nails, comprising applying the composition of claim 1 to said fungal infection or to the skin or tissue surrounding said nails.

21. A method of making a composition of claim 1 comprising dissolving said compound into water in amounts sufficient to result in a pH in the range of about 2.0 to about 4.0 or in an effective concentration and, if necessary, adjusting the pH with a pH adjuster into the range of about 2.0 to about 4.0.

22. The method of making the composition of claim 21 wherein said metal is tin and said halide is fluoride, comprising converting said compound into an oxide thereof before dissolving said compound into said water.

23. The method of making the composition of claim 22 wherein said compound, before conversion into said oxide is stannous fluoride.

24. The method of claim 23 wherein at least a portion of said stannous fluoride is oxidized to stannic oxyfluoride prior to dissolving said compound into said water.

25. The method of claim 24 wherein said compound is dissolved into said composition up to said compound's saturation point.

26. The method of claim 25 wherein said composition has an excess of said compound present as solids together with said saturated solution of said compound.

27. A kit comprising a composition of claim 1 and labeling therefore including at least instructions on the use of said composition in a topical antifungal method.

28. A kit for making the composition of claim 1 comprising a solids component and a liquids component, said solids component comprising components b and optionally d of claim 1, said liquids component comprising components a and optionally c of claim 1, said kit further containing instructions for reconstitution thereof and use of the reconstituted composition as an antifungal topical composition.

29. A kit for making the composition of claim 1 comprising at least one solid component b of claim 1, at least one liquid component a of claim 1, optionally at least one solid component d of claim 1, and optionally one liquid component d of claim 1, and further comprising instructions for reconstituting said composition of claim 1 and use thereof as an antifungal topical composition.

30. A method of treating a dermatological condition comprising applying a composition comprising composition comprising:
(a) water; and
(b) an active member selected from the group consisting of
(i) a first compound capable of generating in aqueous solution, at least one type of halogen ion X and H ion;
(ii) a second compound capable of generating in aqueous solution at least one halide ion X but not a significant amount of H ions in combination with a third compound capable of generating in aqueous solution H ions but not a significant amount of at least one halide ion X; and
(iii) mixtures thereof;
wherein each of said first and second compound comprises a transition metal selected from the group consisting of Ag, Cu, Fe, Ga, Ge, In, Ni, Sn, Ti, Zn, and Zr; and said at least one halide ion X is selected from the group consisting of F, Cl, Br, I, halophosphates of each, and mixtures thereof;
(iv) an alcohol selected from the group consisting of lower monohydric alcohols and lower dihydric alcohols, present in amounts of from 0% up to about 80% by volume based on the final formulation;
(v) optionally a pH adjuster component selected from acids, bases and buffers in amounts sufficient to maintain the pH of said composition in the range of 2.0 to 4.0;
(vi) said composition having a pH of about 2.0 to about 4.0.
wherein said condition is selected from the group consisting of acne, aphthous ulcers, bullous pemphigoid, candida, carbuncles, chiggers, folliculitis, furuncle, herpes simplex, herpes zoster, impetigo, lyme disease, molluscum contagiosum, pfiesteria, pimples, pityriasis rosea, psoriasis, ringworm, scabies, seborrheic dermatitis, seborrhea, tinea cruris, and tinea pedis.

31. The method of claim 30 wherein said condition is psoriasis.

32. The method of claim 30 wherein said first compound is selected from the group consisting of a tin halide and dilute hydrofluoric acid, and said second compound is a non-halide tin compound.

33. A method of treating a dermatological condition comprising applying a composition comprising:
(a) water; and
(b) an active member selected from the group consisting of
(i) a first compound capable of generating in aqueous solution, at least one type of halogen ion X and H ion;
(ii) a second compound capable of generating in aqueous solution at least one halide ion X but not a significant amount of H ions in combination with a third compound capable of generating in aqueous solution H ions but not a significant amount of at least one halide ion X; and
(iii) mixtures thereof;
wherein each of said first and second compound comprises a transition metal selected from the group consisting of Ag, Cu, Fe, Ga, Ge, In, Ni, Sn, Ti, Zn, and Zr; and said at least one halide ion X is selected from the group consisting of F, Cl, Br, I, halophosphates of each, and mixtures thereof;
(c) an alcohol selected from the group consisting of lower monohydric alcohols, lower dihydric alcohols, and lower polyhydric alcohols, present in amounts of from 0% up to about 80% by volume based on the final formulation;
(d) optionally a pH adjuster component selected from acids, bases and buffers in amounts sufficient to maintain the pH of said composition in the range of 2.0 to 4.0;
(e) optionally a thickener selected from the group consisting of organic and inorganic thickening agents;
(f) optionally a surfactant selected from cationic, anionic, nonionic, amphoteric, and zwitterionic surfactants;
said composition having a pH of about 2.0 to about 4.0.
wherein said condition is selected from the group consisting of acne, aphthous ulcers, bullous pemphigoid, candida, carbuncles, chiggers, folliculitis, furuncle, herpes simplex, herpes zoster, impetigo, lyme disease, molluscum contagiosum, pfiesteria, pimples, pityriasis rosea, psoriasis, ringworm, scabies, seborrheic dermatitis, seborrhea, tinea cruris, and tinea pedis.

34. A method of treating a dermatological condition comprising applying a composition comprising an active member selected from the group consisting of
(i) a first compound capable of generating in aqueous solution, at least one type of halogen ion X and H ion;
(ii) a second compound capable of generating in aqueous solution at least one halide ion X but not a significant amount of H ions in combination with a third compound capable of generating in aqueous solution H ions but not a significant amount of at least one halide ion X; and
(iii) mixtures thereof;
wherein each of said first and second compound comprises a transition metal selected from the group consisting of Ag, Cu, Fe, Ga, Ge, In, Ni, Sn, Ti, Zn, and Zr; and said at least one halide ion X is selected from the group consisting of F, Cl, Br, I, halophosphates of each, and mixtures thereof;
wherein said condition is selected from the group consisting of acne, aphthous ulcers, bullous pemphigoid, candida, carbuncles, chiggers, folliculitis, furuncle, herpes simplex, herpes zoster, impetigo, lyme disease, molluscum contagiosum, pfiesteria, pimples, pityriasis rosea, psoriasis, ringworm, scabies, seborrheic dermatitis, seborrhea, tinea cruris, and tinea pedis.

35. The method of claim 34 wherein said composition is in the form of a solution, a suspension, a gel, a lotion, a cream, or an ointment.

36. The method of claim 34 wherein said composition is contained in an impregnated bandage, a topical or transdermal or transcutaneous patch.

37. The method of claim 34 wherein said composition is aqueous.
